(19) United States
(12) Patent Application Publication
(10) Pub. No.: US 2019/0352595 A1
(43) Pub. Date: Nov. 21, 2019

(54) METHOD AND DEVICE ASSEMBLY FOR PROVIDING A LARGE AMOUNT OF CELL CULTURE MEDIUM USING DISPOSABLE COMPONENTS

(71) Applicant: SARTORIUS STEDIM BIOTECH GMBH, Goettingen (DE)

(72) Inventors: Christian GRIMM, Heilbad Heiligenstadt (DE); Mario BECKER, Goettingen (DE); Christian MANZKE, Bovenden (DE)

(21) Appl. No.: 16/475,774

(22) PCT Filed: Dec. 4, 2017
(86) PCT No.: PCT/EP2017/081384

§ 371 Date: Jul. 3, 2019

(30) Foreign Application Priority Data
Jan. 5, 2017 (DE) 10 2017 100 150.6

(51) Int. Cl. C12M 1/34; C12M 1/00; C12M 1/06; C12M 1/12

(52) U.S. Cl. CPC C12M 41/30; C12M 29/26; C12M 41/44; C12M 37/00; C12M 27/02

(57) ABSTRACT

A method for providing a large amount of cell culture medium (10), in particular for cultivating animal cells, for a large reactor system (12) by using single-use components, comprises the following sequence of steps:
a) preparing a concentrated media stock solution (32),
b) filling the concentrated media stock solution (32) into single-use containers (40),
c) transporting the single-use containers (40) filled with media stock solution (32) to the large reactor system (12),
d) aseptically connecting the single-use containers (40) to the large reactor system (12) by means of a single-use hose system (26),
e) processing the media stock solution (32) by supplying ultrapure water (24) and/or additives (44), and
f) supplying the processed media stock solution (32) into the large reactor system (12).

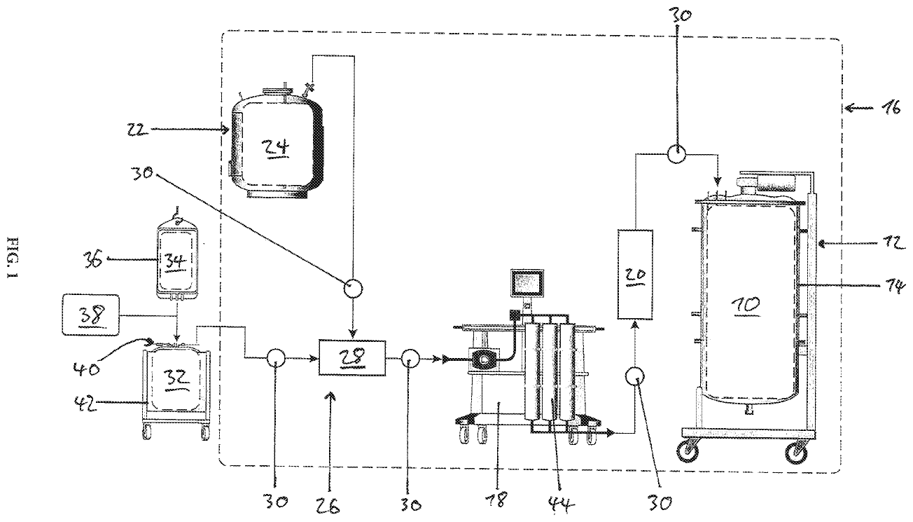

The invention relates to a method for providing a large amount of cell culture medium, in particular for cultivating animal cells, for a large reactor system by using single-use components. The invention furthermore relates to an apparatus arrangement which is configured to carry out such a method.

The trends of recent years in the biopharmaceutical industry increasingly point in the direction of the use of single-use components (SU components). The same no longer are used now only in the field of product and process development, but also in the field of Clinical Trial Manufacturing (CTM) for the approval procedure and even in the commercial “Good Manufacturing Practice” (GMP) in the production of drugs.

In the commercial production, such as for example in the cultivation of animal cells in a bioreactor, two different approaches of the utilization of single-use components substantially are employed. On the one hand, especially in new factories, locations or enterprises especially in Asia, the operators rely on a full single-use approach in which the entire manufacturing chain is covered almost exclusively by single-use components. On the other hand, when classical appliances made of stainless steel are present already, the same in general shall also continue to be used due to the high purchase price. In this case, so-called hybrid installations comprising existing stainless steel components and newly added single-use components are being used. One example is the cultivation of animal cells in a bioreactor. The bioreactor here for example is an existing stainless steel boiler with a volume of 2,000 to 50,000 liters, but the media and buffer production is effected in single-use mixing and storage tanks.

Both in complete single-use installations and in hybrid systems an essential challenge consists in providing the suitable amount of medium for the cell culture in a reactor with a high operating volume of several thousand liters. As in single-use systems no rigid tubing or the like with a large media preparation tank exists or is provided, the medium must be prepared in handleable containers, be stored and be transported to the reactor. These containers normally have a capacity of merely 50 to 1,000 liters. For larger production reactors, a plurality of such containers must therefore be brought to a reactor, be aseptically connected and be supplied to the reaction vessel in the sterile-filtered condition. This mostly is accomplished sequentially and requires a high manual effort when properly connecting and separating the containers. In addition each connection offers a potential risk for a leakage or a contamination of the reactor.

It is the object of the invention to create a method which makes the process of providing a large amount of cell culture medium for a large reactor system by using single-use components more efficient and less risky.

In order to achieve the object, there is provided a method for providing a large amount of cell culture medium, in particular for cultivating animal cells, for a large reactor system by using single-use components, comprising the following sequence of steps:
a) preparing a concentrated media stock solution,
b) filling the concentrated media stock solution into single-use containers,
c) transporting the single-use containers filled with the media stock solution to the large reactor system,
d) aseptically connecting the single-use containers to the large reactor system by means of a single-use hose system,
e) processing the media stock solution by supplying ultrapure water and/or additives, and
f) supplying the processed media stock solution into the large reactor system.

In this way, the media stock solution is provided in concentrated form and only on site at the reactor system diluted to the required mixing ratio. Thus, the volume and the number of the single-use containers which are required for the transport and must each be connected to the reactor system is reduced. Owing to the invention, large amounts of cell culture medium can also be efficiently provided to a reactor system by using single-use components and the risks, for example a contamination of the reactor, can be reduced.

In step a) the concentrated media stock solution can be matched to the volumes of subsequent mixing systems and/or to the reactor volume. In this way, the processing of the media stock solution is simplified, as for example existing mixing systems can be used without any additional modification. Furthermore, matching to the reactor volume allows that the provided number of single-use containers with the concentrated media stock solution can be drained completely into the reactor, whereby the rest of media stock solution, which is obtained as waste, is minimized.

In step a), the concentrated media stock solution preferably has a concentration of 5:1 or higher. The higher the concentration of the media stock solution, the smaller the volume of the media stock solution which is required for preparing a particular solution in the reactor. With a smaller volume, the number of single-use containers required for the transport of the media stock solution also is reduced. This leads to a cost reduction, as fewer single-use containers are consumed and the transport is less costly.

In a preferred embodiment, the concentrated media stock solution in step b) is filled into transportable single-use containers each having a volume of 1 liter to 500 liters, preferably 5 liters to 200 liters. Single-use containers of this size have the advantage that they can be handled with simple means, for example with trolleys and lift trucks. Furthermore, with a fixed concentration of the media stock solution a plurality of single-use containers, in particular of different sizes, can be combined correspondingly in order to exactly provide the required amount of media stock solution for the reactor system.

In another preferred embodiment the following steps are carried out between step a) and step b):
g) filling the concentrated media stock solution into single-use containers, and
h) preparing the media stock solution by supplying ultrapure water and/or additives.

The produced concentrated media stock solution thereby is temporarily stored at the manufacturer in single-use containers and at a later date matched to the individual requirements of a reactor system. This has the advantage that a concentrated media stock solution can be produced in large amounts and later on be used as a basis for various media stock solutions with different requirements. In particular, additives which only have a limited durability or useful life can also be supplied to the media stock solution. Furthermore, the use of single-use containers offers advantages due to a simplified quality assurance, in particular of hygiene.

In step e) of the method of the invention unstable chemical components can be supplied to the media stock solution. By admixing these additives having a short durability only shortly before supplying the media stock solution to the reactor system, the concentration of these time-critical additives can be adjusted easily and effectively in the reactor.

The single-use hose system can comprise a static mixer which dilutes the media stock solution, preferably with ultrapure water, in particular to obtain a 1:1 concentration. By means of the static mixer the media stock solution can be diluted in a simple way to obtain a suitable concentration (in particular inline dilution methods are possible).

According to an advantageous embodiment, the media stock solution is sterile-filtered before step f) of the method of the invention. The risk of a contamination of the reactor system thereby is reduced. The sterile filtration is effected in particular directly before step f) in order to also trap germs which have been introduced in preceding steps. Alternatively, the sterile filtration can also be effected already at an earlier stage, for example on introduction into the single-use hose system.

According to another advantageous embodiment sensors are arranged in the single-use hose system, which are used for controlling at least one of steps e) and f) of the method according to the invention by means of an automation unit. Due to the automated control of these steps a particularly high quality of the processed media stock solution is ensured.

The object of the invention also is achieved by an apparatus arrangement for providing a large amount of cell culture medium, which is configured to carry out the method according to the invention. With respect to the advantages of the apparatus arrangement of the invention reference is made to the corresponding explanations on the method according to the invention.

In a preferred embodiment the apparatus arrangement according to the invention comprises an electronic monitoring and control device, in particular a SCADA system, which is configured to adjust the concentration of the concentrated media stock solution in step a). The automated adjustment makes a manual intervention superfluous.

What is particularly advantageous is the use of sensors for determining the pH value and/or the conductivity of the concentrated media stock solution. The monitoring and control device then can control the adjustment of the concentration of the concentrated media stock solution on the basis of the data of the sensors. Such an automated control by taking account of the parameter values provided by the sensors continuously provides exact, reproducible results without a user having to make adjustments or readjustments in ongoing operation.

For the automated control of the processing of the media stock solution and/or the supply of the processed media stock solution into the large reactor system an automation unit preferably is provided in the reactor installation. Via the automation unit additives can be metered to the diluted media stock solution automatically and with a high accuracy.

According to an advantageous aspect of the invention it is possible by means of aseptic connections and a static mixer, which dilutes the media stock solution, preferably with ultrapure water, in particular to obtain a 1:1 concentration, to realize a single-use hose system for the reactor installation, which in particular can be completed by a sensor for determining the flow rate. Aseptic connections here are understood to be hose connections or connections of single-use components which can be realized by means of aseptic connectors or e.g. by welding together TPE hoses or by similar measures.

Preferably, the single-use containers can be sterilized by gamma radiation, whereby they can be degerminated with little effort before filling and can be disposed of professionally after emptying.

The reactor system can comprise a single-use bioreactor made of a suitable plastic material or a reactor made of stainless steel. This means that the method is also suitable for hybrid reactor systems with existing stainless steel appliances.

Further advantages and features can be taken from the following description in conjunction with the attached drawing. The only FIGURE shows a schematic representation of the used appliances and the sequence of the method according to the invention.

The FIGURE schematically shows the apparatus arrangement according to the invention and the method according to the invention, by means of which a large amount of cell culture medium 10 in the form of a stock solution is provided in a large reactor system 12.

The large reactor system 12 comprises a reactor 14 and is part of a reactor installation 16, which furthermore comprises an automation unit 18, a sterile filtration device 20 as well as a WFI (Water for Injection) installation 22, which provides ultrapure water 24. The reactor system 12, the automation unit 18 and the WFI installation 22 are connected to each other via a single-use hose system 26 which includes a static mixer 28 and a plurality of sensors 30.

The sensors 30 in particular are provided for determining the flow rates, the pH values and the conductivity. The pressure determination also is of interest, above all with regard to a safety shut-off. Furthermore, sensors 30 can be provided for optical spectroscopic identification methods and quantitative analysis methods.

The reactor 14 is a stainless steel reactor with an operating volume of 10,000 liters, which in contrast to the single-use components employed in the method is further utilized for subsequent productions. The reactor installation 16 therefore is a hybrid installation.

In a first step of the method according to the invention a concentrated media stock solution 32 is produced outside the reactor installation 16, for example at a media manufacturer. The concentrated media stock solution 32 is obtained from powdery and/or liquid components 34 which are provided in tubular bags 36.

The concentrated media stock solution 32 can be produced in a single-use mixing system (not shown).

The concentration of the concentrated media stock solution 32 is adjusted via a SCADA (Supervisory Control and Data Acquisition) system 38 and controlled by means of sensor data of the pH value and the conductivity. The concentrated media stock solution 32 is produced with a particularly high concentration of 10:1, i.e. each unit volume of the media stock solution 32 contains only very little water.

In a next step the concentrated media stock solution 32 is filled into sterile single-use containers 40. The single-use containers 40 each hold a volume of about 200 liters and can be transported by a person using a trolley 42. Furthermore, the single-use containers 40 can be sterilized by gamma radiation, whereby they can be sterilized in a simple way before filling.

Subsequently, the single-use containers 40 filled with concentrated media stock solution 32 are temporarily stored, before they are transported to the reactor system 12.

In an optional step the concentrated media stock solution 32, in particular shortly before the transport to the reactor system 12, is diluted further and/or further additives are admixed to the concentrated media stock solution 32 in order to comply with the individual requirements of the reactor system 12 or the user of the media stock solution 32. The additives in particular can include constituents with a limited durability or useful life, which is why they are not suitable for a temporary storage and therefore are supplied shortly before the transport.

Alternatively, further additives can also be admixed to the concentrated media stock solution 32 directly during the production, in particular when the properties of the concentrated media stock solution 32 do not change disadvantageously until being supplied into the reactor system.

After this preparation of the concentrated media stock solution 32, the same is again filled into corresponding sterilized single-use containers 40.

During the production in the first step or in the optional step during the preparation of the media stock solution 32, the concentration of the concentrated media stock solution 32 is matched to the volume of the reactor 14 and of the automation unit 18 so that the required amount of cell culture medium 10 can be provided by the concentrated media stock solution 32 without any modification of the reactor installation 16.

After filling the produced or prepared concentrated media stock solution 32 and after the optional temporary storage, the single-use containers 40 filled with the media stock solution 32 are transported to the reactor system 12.

In a subsequent step, the single-use containers 40 are aseptically connected to the reactor system 12 by means of the single-use hose system 26.

The media stock solution 32 is processed in a next step. The media stock solution 32 here is diluted with ultrapure water 24 from the connected WFI installation 22 by means of the static mixer 28 to obtain a 1:1 concentration. In addition, additives 44 are metered to the diluted media stock solution 32 via the automation unit 18. These additives 44 in particular contain unstable chemical components which due to their short useful life are admixed to the media stock solution 32 only at this time in order to be able to ensure a particular concentration of these additives 44 in the reactor system 12.

Of course, in this step less critical additives 44 can also be supplied to the media stock solution 32 or additives 44 which are available only to the user of the media stock solution 32, for example due to licences or safety regulations.

Subsequently, the processed media stock solution 32 in a further step is introduced into the sterile filtration device 20 and sterile-filtered.

In the last step, the sterile-filtered processed media stock solution 32 is supplied to the reactor system 12 and now is available there for example for the cultivation of animal cells.

The automation unit 18 monitors all the aforementioned steps which take place within the reactor installation 16 and controls the entire process by means of the sensor data which are transmitted to the automation unit 18 by the sensors 30. Thus, a high process safety is ensured.

In this way, the method according to the invention allows to provide a large amount of stock solution in a large reactor system 12, which in particular is part of a hybrid installation, and at the same time to reduce the transport expenditure and the risk of a contamination of the reactor system 12. In the exemplary embodiment described above 10,000 liters of cell culture medium are provided by the method of the invention by means of fewer single-use containers each with a capacity of only 200 liters.

1. A method for providing a large amount of cell culture medium for a large reactor system by using single-use components, comprising a sequence of steps as follows:
a) preparing a concentrated media stock solution,
b) filling single-use containers with the concentrated media stock solution,
c) transporting the single-use containers filled with the media stock solution to the large reactor system,
d) aseptically connecting the single-use containers to the large reactor system by means of a single-use hose system,
e) processing the media stock solution by supplying ultrapure water and/or additives, and
f) supplying the processed media stock solution into the large reactor system.

2. The method according to claim 1, characterized in that in step a) the concentrated media stock solution is matched to the volumes of subsequent mixing systems and/or to the reactor volume.

3. The method according to claim 1, characterized in that in step a) the concentrated media stock solution has a concentration of 5:1 or higher.

4. The method according to claim 1, characterized in that in step b) transportable single-use containers are filled with the concentrated media stock solution wherein the transportable single-use containers each having a volume of 1 L to 500 L.

5. The method according to claim 1, characterized in that between step a) and step b) the following steps are carried out:
g) filling the concentrated media stock solution into single-use containers, and
h) preparing the media stock solution by supplying ultrapure water and/or additives.

6. The method according to claim 1, characterized in that in step e) unstable chemical components are supplied to the media stock solution.

7. The method according to claim 1, characterized in that the single-use hose system comprises a static mixer which dilutes the media stock solution, to obtain a lower concentration.

8. The method according to claim 1, characterized in that the media stock solution is sterile-filtered before step f).

9. The method according to claim 1, characterized in that in the single-use hose system sensors are arranged, which are used for controlling at least one of the steps e) and f) by means of an automation unit.

10. An apparatus arrangement for providing a large amount of cell culture medium, characterized in that the apparatus arrangement is configured to carry out the method according to claim 1.

11. The apparatus arrangement according to claim 10, characterized by an electronic monitoring and control device, which is configured to adjust the concentration of the concentrated media stock solution in step a).

12. The apparatus arrangement according to claim 11, characterized by sensors for determining the pH value and/or the conductivity of the concentrated media stock solution, wherein the monitoring and control device is configured to control the adjustment of the concentration of the concentrated media stock solution on the basis of data of the sensors.

13. The apparatus arrangement according to claim 10, characterized by an automation unit for automated control of the processing of the media stock solution and/or supply of the processed media stock solution into the large reactor system.

14. The apparatus arrangement according to claim 10, characterized by a single-use hose system with aseptic connections, and a static mixer which dilutes the media stock solution.

15. The apparatus arrangement according to claim 14, characterized by a sensor for determining flow rate.

16. The apparatus arrangement according to claim 10, characterized in that the single-use containers can be sterilized by gamma radiation.

17. The apparatus arrangement according to claim 10, characterized in that the reactor system comprises a single-use bioreactor made of a suitable plastic material.

18. The apparatus arrangement according to claim 10, characterized in that the reactor system comprises a reactor made of stainless steel.

19. The method for providing a large amount of cell culture medium to a large reactor system for cultivating animal cells.
